(19) United States
(12) Patent Application Publication
(10) Pub. No.: US 2019/0373426 A1
(43) Pub. Date: Dec. 5, 2019

(54) METHOD AND APPARATUS FOR HYPER-LOCAL TESTING AND PERSONALIZED ANALYSIS OF CHEMICAL COMPOSITIONS AND ENVIRONMENTAL CONDITIONS

(71) Applicant: SPRIMO, INC., Santa Clara, CA (US)

(72) Inventors: Ray COMBS, San Jose, CA (US); Jocelyn KING, Los Gatos, CA (US); Linda HOLROYD, Mountain View, CA (US); Curt WARD, Fremont, CA (US)

(21) Appl. No.: 16/432,757

(22) Filed: Jun. 5, 2019

Related U.S. Application Data
(60) Provisional application No. 62/680,939, filed on Jun. 5, 2018.

(51) Int. Cl. H04W 4/38; G01N 33/00; H04L 29/08; G01N 33/18; H04R 29/00

(52) U.S. Cl. CPC H04W 4/38; G01N 33/0065; H04R 29/008; G01N 33/004; G01N 33/18; H04L 67/12

(57) ABSTRACT

A method and apparatus for testing and monitoring of personal, hyper-local chemical composition or environmental conditions is accomplished when a user or group of users gather data utilizing the combination of a sensor-extensible apparatus attached to one or more mobile communications devices, employing the associated mobile computing operating systems and network, in order to capture and transmit the relevant data to a cloud computing complex for storage, analysis and reporting. The cloud computing complex is configured to validate data by gathering corroborating evidence of location and reading accuracy, and provide a recommendation to a user or group of users within a threshold confidence level.

### FIELD OF THE INVENTION

The field of the invention is cloud-based systems and mobile communications devices with sensor modules, for testing, monitoring, and analyzing environmental conditions.

### BACKGROUND

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Collecting and processing data from remote sensing devices is well known. Typically in large area systems for air or water monitoring, a remote sensor remains in a fixed location, senses a particular condition or chemical composition for the given location, and by way of a network, communicates the sensed condition to some back end computing system for storage and retrieval. The resulting data may be analyzed and warnings produced and targeted for a number of people in a building, region, city or state.

For example, in outdoor weather or air quality monitoring systems, a remote sensing device is designed to monitor specific chemical compositions or environmental conditions such as temperature, humidity, wind, barometric pressure, ozone, carbon monoxide, formaldehyde, particulate matter, pollen count, lead, sulfur dioxide, and other conditions. The sensed weather conditions are then provided to a processing device, which compiles the data to describe current conditions and transmits this data to a central computing system in order to predict weather changes, and issue warnings when the weather, hazardous chemical levels or general environment is dangerous in a given area. A typical weather sensing system may include only a few pre-defined, fixed types of sensing devices for an entire region, city or state.

Other remote sensors may be used along rivers or within community water supply systems to measure water levels, flow rates, water quality, chemical composition, acidity, chlorine concentrations, etc.

Indoors sensing devices may be used within buildings to monitor general air quality, temperature, humidity, gases, pollutants, oxygen levels, noise levels, light levels, electromagnetic signals, etc. These devices are typically fixed in location within a building or room in order to capture data related to the specific building or room. Other sensing devices within buildings may be attached to water pipes or within the flow of water for testing pressure, flow, noise, leaks, and chemical composition within the water such as lead levels, zinc, or other metals.

Regardless of the type of remote sensing, the remote sensing devices are typically fixed in location, are typically pre-defined for certain chemical composition or environmental conditions to be tested and are relatively few in number when compared to the number of mobile computing devices readily available to a geographical area. The fixed location and relatively few amount of sensing devices results in a lower resolution of data. Additionally, the data collected typically relates to areas or regions shared by multiple people, such as a city, a neighborhood, a building, an office or a room and do not report on the hyper-local, i.e. personal environment occupied by each individual within a room, building, neighborhood or city. Additionally, fixed location sensing devices do not take into account the potentially rapid changing of air and water and environmental conditions in a remote location where forest fires, volcano eruptions, chemical spills, military conflict or industrial accidents can quickly change potential human exposures to chemical levels and hazardous environments.

Additionally, centralized data collecting systems that warn on unhealthy environmental conditions—such as air quality, water quality, lead levels, sunlight levels, ozone levels, pollen counts, air humidity, poisonous gases, particulate levels, smoke, chemical compositions, and so forth, capture data and report on generally accepted fixed thresholds. The fixed thresholds are usually standardized by governments or institutions and established based on average human sensitivity to the particular chemicals or environmental conditions. For individuals who are more sensitive to a chemical or an environmental condition, the nominal threshold may be too high. Thus, for these people, the normal reports and warning levels may not be appropriate to protect their safety and good health.

Therefore, a need exists for methods and sensor-extensible apparatus that allow for hyper-local, mobile, personally detailed collection of data regarding chemical exposures and environmental conditions, with customizable warning processes which allows for personalizing of feedback and personalized warning levels regarding exposure to chemical compositions or environmental conditions.

### SUMMARY OF THE INVENTION

The inventive subject matter includes a cloud-based computing infrastructure coupled with a plurality of mobile communication devices. The mobile communication devices are equipped with sensor modules configured to measure environmental conditions such as chemical compositions disposed in the environment. The contemplated systems and methods facilitate the gathering of large amounts of data based on a crowd-sourcing methodology to improve data analytics and predictive data extrapolations. The mobile communication devices are also configured with validation and augmentation software modules to gather additional corroborating data associated with each sensor module reading, and to improve reliability and confidence in each reading. Corroborating data can be used to validate both the accuracy of the readings and the accuracy of the location data associated with each reading. These modules are also configured to prompt users to provide observations about environmental conditions and provide imagery and/or sound recordings.

The cloud-based computing infrastructure further includes a recommendation module for generating recommendations such as alarms or warnings, reports, trends, and predictions. The recommendations can be highly personalized to a particular user or locality within a larger region based on the large amount of sensor readings and detailed hyper-local validation/augmentation information. The recommendation module is also configured to consult with government and institutional databases that provide additional environmental condition data, as well as rules, regulations, and best practices. The large data collection and reports can be commercially provided to municipalities, agencies, and private organizations with a confidence score representing a high degree of data integrity and trustworthiness using the validation and augmentation information. The aggregation of large hyper-local data can also be used to better understand historical trends in environmental conditions and the associated causes and affects related to environmental changes.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of an embodiment of a cloud-based system for hyper-local testing of environmental conditions.

FIG. 2 is an illustration of an embodiment of a mobile communications device and a sensor module.

FIG. 3 is a schematic of an embodiment of a cloud-based computing infrastructure.

FIG. 4 is an illustration of an embodiment of a data collection report displayed on a mobile communications device.

FIG. 5 is an illustration of another embodiment of a data collection report displayed on a mobile communications device.

### DETAILED DESCRIPTION

The following description includes information that may be useful in understanding the present invention. It is not an admission that any of the information provided herein is prior art or relevant to the presently claimed invention, or that any publication specifically or implicitly referenced is prior art.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

One should appreciate that the disclosed techniques provide many advantageous technical effects including testing and monitoring hyper-local chemical compositions and environmental conditions, and providing predictions, warnings, and recommendations.

The following discussion includes example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Generally, the present invention provides a method and apparatus for testing, monitoring and analyzing chemical and environmental conditions utilizing a multiple of public and private users of a wireless communications system in order to capture and analyze data to a central cloud-computing environment. The data can then be analyzed and reported on or other actions taken as appropriate, as determined through the use of intelligent software algorithms and software-based decision making.

This is accomplished when a user, or group of users, utilize commonly available mobile communications devices with mobile operating systems and wireless networking capabilities such as smart phones, tablets or laptop computers, and attach to the mobile communications devices a customizable apparatus that specializes in sensing the desired chemicals or environmental conditions within the desired matter to be tested.

The sensor-extensible apparatus is designed to accept a plurality of interchangeable chemical or environmental sensors, depending on the desired matter to be tested, the testing to be performed and the data to be collected. The apparatus consists of two primary components—a low cost sensor module, and a cell phone or other base unit that accepts the attachment of one or more sensor modules.

As there are thousands of potentially desirable chemicals or environmental conditions that could be tested, the sensor-extensible apparatus is designed to accept simplistic, low cost sensor modules using a proprietary connection type and proprietary communications protocol that (a) identifies the sensor module by type or serial number (b) the type or category of data the specific sensor module is designed to collect and (c) may provide power to the sensor module as necessary, either or both of the apparatus base unit and the sensor modules may include internal computing capabilities, including but not limited to data processing, memory and storage, wired or wireless networking capabilities, communications ports, battery, software or firmware, plus controls and displays as necessary to operate the apparatus.

The resulting apparatus base unit plus sensor modules may attach to the mobile communications device by wired or wireless communications channels. Wired connections may include standards-based communications cables or proprietary cabling, depending on the compatibility of the mobile communications device. For example, many mobile communications devices use standards-based USB (Universal Serial Bus), fiber, optical or other cables. Others may require proprietary connectors and protocols such as Apple'S™ Lightning™ connection. Wireless communications channels may include one or more short-range connections such as Bluetooth™, WiFi™, ZigBee™, Infrared™, etc.

The capturing of data may include hyper-local physical data such as, but not limited to, the type of matter being tested (gas, liquid, solid), specifically targeted chemicals, time and location, air or water quality, moisture, pollen, metal concentration, light concentration, sound or radio frequencies and strengths, etc.

Augmentation of the physical data may include imagery or sound obtained from the mobile communications device or by other means. For example, when physical data is collected, a photo may be superimposed on a report, chart or real time readings in order to validate the location or environment where the physical data was collected. This type of augmentation provides another level of evidence of the situation or location where the physical data was collected. Sound recordings associated with the collected physical data may add extra human-recognizable information, such as human voices, animal vocalizations, or environmental noises, which enhances the physical data.

Also, the captured hyper-local physical data may be augmented by human generated input in order to enhance the quality of the captured physical data before transmitting the data to the cloud-computing complex. As the data is collected, the mobile communications device through a software application and user interface, may prompt the user for additional information or validation or fine tuning of human-discernible information such as mapping information, postal codes and addresses, names of nearby buildings or monuments, visual, olfactory or other sensory observations. Additionally, user names, ages, gender or other personal attributes of the user performing the testing may be captured as part of the data validation process.

An exemplary case of augmenting the data would be where the individual is exposed to the measured air chemical compositions or environmental conditions, the user is prompted to add a photo or video or audio recording of the environment or surroundings as a form of validation of the data. Additionally, he is prompted for additional information, such as responding to questions or filling in a form, to be associated with the captured sensing data. This personalized and augmented data is then stored to the cloud services and attributed to a particular user with multiple data samples over a period of time (minutes, hours, days, weeks, etc.). Such personalized and augmented data and records can then be used as input to create reports or alarms specifically customized for the individual and the individual's sensitivities. The reports may also include descriptions of potential human side effects of exposure to certain chemicals or environmental conditions.

Another exemplary case is the ad hoc measurement of specific air elements such as CO₂, in a work space. Such an example can be used to determine the healthiness of a work environment, in which an individual walks around and takes air samples over a period of time to determine periods where and when CO₂ levels are highest or lowest. The data can be augmented with photos or videos showing working environments and worker attitudes or attentiveness or alertness. Such rich media would then augment the sensing data for increased validity.

Another exemplary case of augmenting the data would be where the individual is consuming drinking water from unknown sources with unknown quality, where the measured chemical compositions are taken of the water before or after drinking. The user can be prompted to add a photo or video of the environment or water source, for example in a stream or public drinking fountain or cup of water in a restaurant, as a form of validation of the data. This is particularly interesting if the quality of the drinking water being consumed is unknown and the user has particular sensitivities that need to be tracked over time. Reports or charts can be made of levels of chemicals or contaminants consumed over time, showing consumption rates and accumulated totals for the individual.

The captured data may then be transmitted to the cloud-computing complex and may be coupled with other environmental data collected by governments or other agencies as a comparison of the hyper-local values to the more generalized population values.

The resulting cloud-based data may then be analyzed in real time or over longer periods of time as necessary to provide trending information, reports, charts or alerts and warning messages on a user-specific, group-specific or location-specific basis. The resulting analysis, including alert levels and the content of warning messages can change as new data creates new trending information.

The types and frequency of information or messages generated may be based on individual user preferences, group settings or governmental standards and may be delivered to individuals, private groups of users or transmitted publicly on a regional or governmental basis. Individuals can adjust their personal preferences or settings based on their physiological sensitivities or on their individual needs or desires.

These personalized settings and custom reports based on these settings can then be stored and processed using a centrally located computing system and compared with personalized settings and data from multiple individuals or multiple public or private groups in order to provide predictions and warnings for other individuals with similar physiological sensitivities. These other individuals with similar sensitivities who benefit from these predictions, warnings or reposts may be associated with a testing group or may be in the public domain. Additionally, publicly available data may be incorporated into the overall analysis to provide validation of generated predictions, warnings and reports and thereby enhancing the quality of these predictions, warnings and reports.

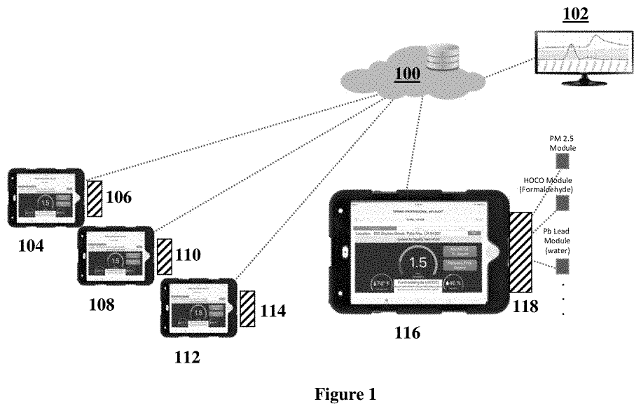

FIG. 1 shows a cloud-based computing infrastructure 100 communicatively coupled with a plurality of mobile communication devices 104, 108, 112, and 116. The mobile communication devices 104, 108, 112, and 116 are communicatively coupled with the cloud-based computing infrastructure 100 via a wired or wireless connection suitable for sending and receiving data. Contemplated connections include local area networks (LAN), wide area networks (WAN), and wireless protocols such as Bluetooth, BLE, ZigBee, Z-Wave, 6LoWPAn, Thread, WiFi-ah, 2G (GSM), 3G, 4G, LTE, NB-IoT, 5G, NFC, RFID, SigFox, LoRaWAN, Ingenu, Weightless-N, Weightless-P, Weightless-W, ANT, ANT+, DigiMesh, MiWi, EnOcean, Dash7, and WirelessHART.

Each of the mobile communication devices 104, 108, 112, and 116 has a sensor module 106, 110, 114, and 118, respectively, for collecting environmental data. Sensor module 118 comprises three different sensors for measuring different environmental characteristics: (1) PM 2.5 Module, (2) HOCO Module (formaldehyde), and (3) Pb Lead Module (water). The mobile communication devices 104, 108, 112, and 116 also have a user interface for displaying reports and for inputting additional information regarding environmental conditions. The mobile communication devices 104, 108, 112, and 116 are also equipped with GPS hardware, a camera for video recording and taking still pictures, and a microphone and an audio recorder for recording sound.

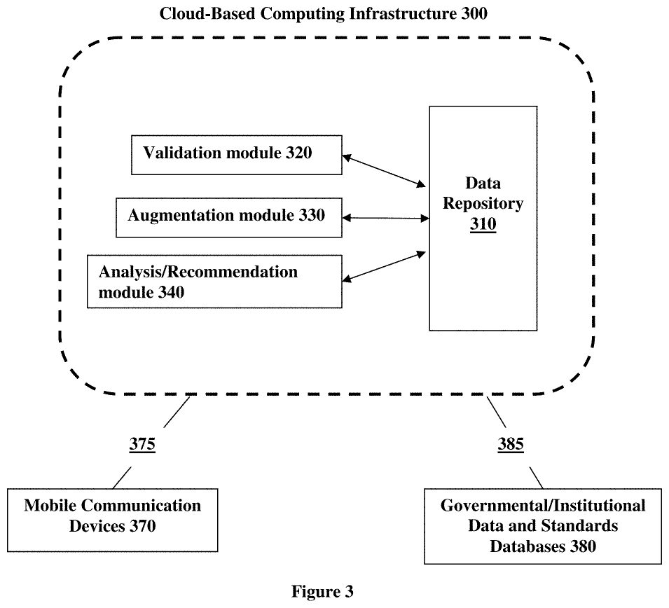

Cloud-based computing infrastructure 100 includes a database, processor, and executable software instructions for collecting sensor data, analyzing information, and generating reports and recommendations. Cloud-based computing infrastructure 100 also includes a display 102 for viewing and analyzing data, as well as viewing reports and providing additional information. The various features of a preferred embodiment of a cloud-based computing infrastructure will be described in greater detail below within the context of FIG. 3.

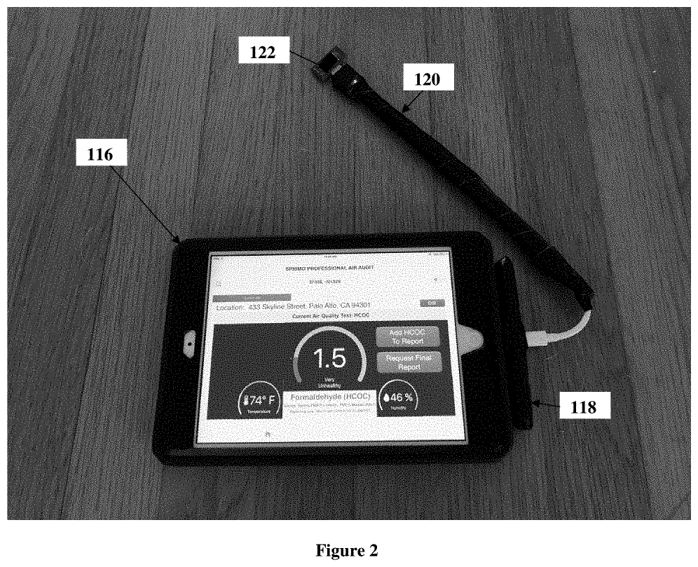

FIG. 2 shows the mobile communication device 116 and sensor module 118 of FIG. 1. Sensor module 118 is connected with a probe 120 having a sensor 122 configured to measure formaldehyde content in a substance or fluid. Probe 120 can include its own portable battery supply; however it is contemplated that a battery in device 116 can also be used to supply power to probe 120. The correct amount of power to be supplied can be determined by a plug-and-play handshaking protocol that identifies the specific sensor module and its power requirements. A user can collect data regarding formaldehyde content in a fluid by placing sensor 122 in the fluid. The display on mobile communication device 116 shows a reading, in this case “1.5” and an indication regarding the reading, e.g., “very unhealthy.” The display also shows a location and time of the reading, and other environmental conditions such as temperature and humidity. The display also provides a software tool for adding the collected data to a report (“Add HCOC to report”) and for generating a report (“Request Final Report”). The display can also be configured to prompt the user for validating information to validate the accuracy of the reading as well as the accuracy of the location information associated with the reading. For example, the interface can be configured to prompt the user to provide a picture or video recording of the location where the reading was taken. The interface can also be configured to request user observations about the environment, such as to describe the color or smell of the medium being measured. The user may also be prompted to take a second or third additional reading to determine a standard deviation of the data specific to that location.

FIG. 3 shows a schematic of a cloud-based computing infrastructure 300 for collecting and analyzing environmental data. Cloud-based computing infrastructure 300 includes a data repository 310, which can comprise one or more non-transitory storage mediums for storing data electronically. Data repository 310 can comprise a plurality of databases distributed across different locations and communicatively coupled via a wired or wireless network. It is also contemplated that data repository 310 could comprise a central database having one or more hard drives or servers located at the same physical location. In either case, data repository 310 is configured to store data collected in a crowd-sourced fashion from a plurality of different mobile communication devices 370 and/or users. The mobile communication devices 370 are communicatively coupled with the cloud-based computing infrastructure 300 via a connection 375. Connection 375 can be wired or wireless, as previously described, and can include encryption and/or security features to maintain data integrity and reliability. Cloud-based computing infrastructure 300 is also communicatively coupled with governmental and/or institutional data and standards databases 380 comprising environmental data, rules, regulations, and best practices. Information from databases 380 can be collected and stored in data repository 310 for analysis and for generating recommendations.

Cloud-Based Computing Infrastructure 300 also includes a validation module 320 comprising executable software instructions configured to validate the accuracy of data collected from the mobile communications devices 370. For example, validation module 320 can be configured to compare new readings to past readings at that same location and determine whether any anomalies in data trends have occurred. Validation module 320 can also be configured to compare new readings from different users and devices at that location, or at nearby locations, and look for data anomalies. The anomalies can be flagged for a human user, such as a data manager, to review and determine whether the new readings are trustworthy or should be discarded. For example, sound recording could be analyzed to determine the level of noise pollution present from airplanes landing at a nearby airport. This data could also be used to corroborate location of a reading or data point. Sound recordings could also be analyzed to determine the presence and/or level of wild life such as birds.

Validation module 320 can be further configured to validate location data associated with readings. For example, validation module 320 can be configured to prompt the user of a particular mobile communication device 370 to capture an image, video recording, and/or sound recording of their environment at the time a reading or measurement is taken to further corroborate GPS location information from the device. An image recognition module and/or sound recognition module can be used in combination with the validation module 320 to analyze the image, video, and/or sound recordings and to identify unique characteristics in the information to corroborate the location data.

Cloud-Based Computing Infrastructure 300 also includes an augmentation module 330 for collecting additional environmental information that can be associated with the reading and sensor data collected from the sensor modules of the mobile communication devices 370. For example, the augmentation module 330 can be configured to prompt the user for observations about the air quality or water quality being measured with the sensor module. The user may be prompted to describe the color or smell of the location, as well as any allergies or body symptoms felt or experienced at the location.

Analysis/Recommendation module 340 is configured to analyze the readings and sensor module data collected from the mobile communication devices 370 and make a recommendation. The recommendation can include an alarm or warning, an indication of quality level, a historical report on environmental conditions for a particular location, a reliability report showing a confidence level of the data collected, and graphs showing historical trends and predicted projections of future conditions.

Analysis/Recommendation module 340 can utilize the data from Governmental/Institutional Data and Standards Databases 380 in addition to sensor module data to make a recommendation. For example, a recommendation could include a comparison of environmental conditions to a government safety regulation, and provide a warning based on the collected sensor readings. Analysis/Recommendation module 340 is also configured to analyze augmentation data and validation data gathered from augmentation module 330 and validation module 320. The recommendation can be provided to a user or group of users via a display of the mobile communication devices 370, via paper printouts, or some other device or method suitable for communicating information.

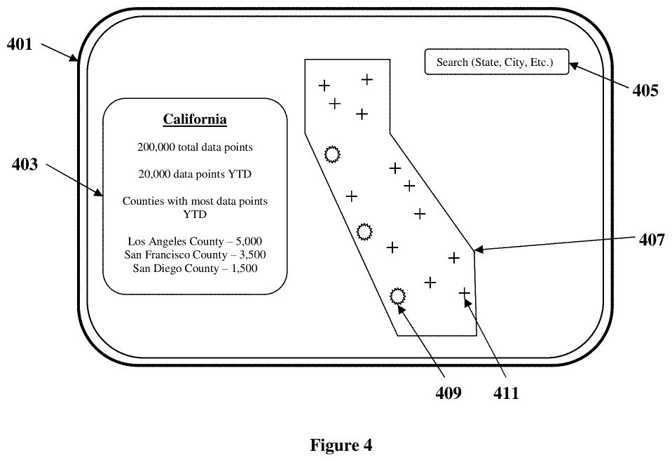

FIG. 4 shows one example of a report 401 displayed on a mobile communication device 370. Report 401 includes a map 407 showing a particular area, in this case the state of California, with indicators 409 showing single readings and indicators 411 showing clusters of readings. Indicators 409 and 411 represent where different types of readings of environmental conditions have been collected within the geographical region displayed in map 407. Search field 405 provides a text description of the geographical area, including the name of the area and GPS or latitude/longitude coordinates. A user can input new text into search field 405 to find and display readings in different regions.

Report 401 also has a summary section 403 that provides an overview of the data collected for the region displayed in map 407. For example, summary section 403 can be configured to show the total data points collected for that area, the total data points for the current year to the present date, counties or sub-regions within map 407 having the most data points. Summary section 403 can also be configured to show levels of environmental conditions, such as average readings, low/high readings, historical trends, and other data analytics and/or recommendations of interest to the user.

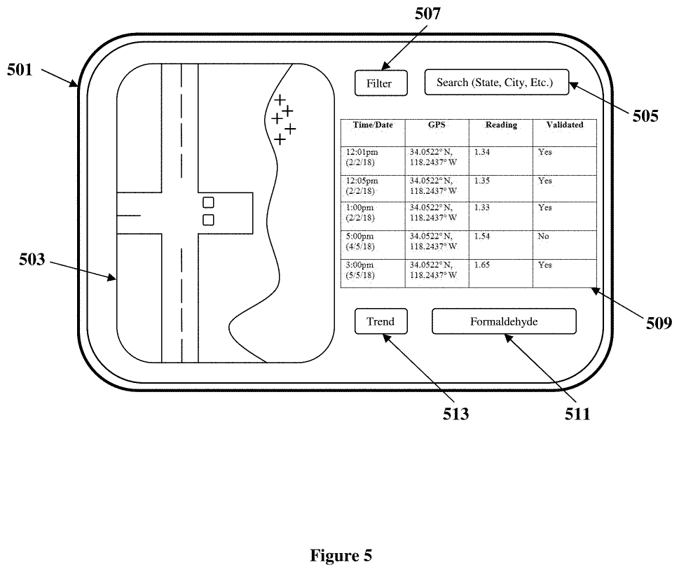

FIG. 5 shows a report 501 displayed on a mobile communications device 370. Report 501 is configured to show hyper-local readings for a zoomed-in area of map 407 in FIG. 4. Map 503 is a satellite view of a zoomed-in portion of map 407 showing the geographical features of the area, including a street and water front. Map 503 also includes indicators (e.g., cross marks) overlaid on the satellite view showing where readings have been taken. Table 509 shows a list of the readings associated with the indicators, including their associated data (time/date, GPS, validation status). A user can select one reading to view all augmentation data associated with that reading, including any pictures, videos, and/or sound recordings, as well as human-generated information such as observations and comments. The list can be ordered and/or filtered based on time/date, reading, location, validation status, or any other criterion associated with each data point using filter button 507. Trend button 513 can be used to display analytics and trends for that particular region. Environmental condition button 511 allows the user to select the type(s) of reading(s) that are shown in map 503. Search field 505 displays the name of the location being displayed and also allows the user to input and search for a new area.

The inventive systems and methods described herein include a mobile communications system having a cloud-based computing infrastructure and a plurality of mobile communications devices with attached sensor-based apparatus. A contemplated method for testing and monitoring chemical compositions and environmental conditions can comprise one or more of the steps of: a) enhancing the physical sensing capabilities of mobile communications devices with sensor-extensible attached apparatus; b) capturing large amounts of physical data, including chemical compositions or environmental conditions, through the use of individual users or crowd-sourcing techniques; c) physically sensing chemical compositions or environmental conditions by one or more of the plurality of communications devices to produce information about the sensed chemical level or environmental; d) capturing the location where the physical sensing was obtained and time the physical sensing was obtained by common methods of determining a location through the use of location based services inherent with mobile communications devices and networking systems; e) augmenting the physical sensing data and information with imagery or sound recordings obtained from the mobile communications device or other means; f) augmenting the physical sensing data with human generated input, both at the time of capturing the sensing data and afterwards through use of software prompting; g) manually validating or refining the captured physical sensing data, location data or other environmental data through use of software prompting to the human through the use of maps, charts, query forms or the like; h) providing, over a mobile communications system and associated networking infrastructure, the physical data and information about the sensed chemical compositions and environmental conditions to a centrally located cloud based computing system; i) storing, processing and analyzing of the obtained physical data and augmented information within a cloud-computing environment; j) enhancing or otherwise augmenting the captured physical data and information by inserting or otherwise comparing the physical data and information with data obtained from outside sources such as government websites; k) providing at least a selected portion of the analyzed data and information to at least one user of the resulting method and apparatus; l) providing specific general alarms, reports or warnings to one or more users based on government standards; m) providing general alarms, reports and warnings to the public affected by adverse changes in local environments, based on government standards; n) providing customized general alarms, reports and warnings to interested users or groups of users affected by adverse changes in local environments, based on the user's specific areas of interests and preferences; o) providing predictions of environmental conditions and physical data based on extrapolations of the large amounts of data captured by users and sensor-based communications devices over time; p) providing recommendations for techniques or processes to correct adverse conditions based on generally accepted methods for correcting such adverse conditions; and q) determining the most effective methods for correcting detected adverse conditions by analyzing large amounts of the physical data captured over time, particularly before and after applying specific correction methods to specific conditions over a large area and a large number of users and groups of users.

In other aspects, a method of crowd-sourcing data for an environmental parameter, using first and second hand-held mobile devices of first and second human users, respectively, is described. The method can comprise the steps of: (i) providing a first sensor to the first human user, the first sensor configured to sense a first value for the environmental parameter; (ii) providing the first mobile device with software that receives the first value, and communicates to a data repository: (a) first data derived from the first value; (b) first location information corresponding to the first value; and (c) at least one of a first image and a first sound recording corresponding to the first value; (iii) providing a second sensor to the second human user, the second sensor configured to sense a second value for the environmental parameter; and (iv) providing the second mobile device with software that receives the second value, and communicates to the data repository: (a) second data derived from the second value; (b) second location information corresponding to the second value; and (c) at least one of a second image and a second sound recording corresponding to the second value.

The step of providing the first sensor to the first human user can comprise either directly or indirectly selling the first sensor to the first human user. The location information corresponding to the first value is preferably current within five minutes of the first mobile device using the software to receive the first value. The first image and the first sound recording are also preferably current within five minutes of the first mobile device receiving the first value. The first mobile device preferably communicates the second data to the data repository within five minutes of the first mobile device receiving the first value. In this manner, data reliability is enhanced and commercial value of the data is increased.

It is contemplated that the data repository can be distal to the first mobile device when the first mobile device communicates the first value to the data repository. The method above can further include the step of assisting a third-party to use information stored in the data repository to provide the first human user with a report that characterizes a health aspect of an environment of the first human user as a function of at least one of the first value and the first data. The report can be directly or indirectly sent to the first human user, characterizing a health aspect of an environment of the first human user as a function of at least one of the first value and the first data. The report additionally can characterize the health aspect of the environment of the first human user as a function of (a) the first location information and (b) at least one of the first image and the first sound recording. The method can further comprise directly or indirectly sending to the first human user a personal history report that depicts time variance of a health aspect of an environment of the first human user as a function of at least one of the first value and the first data.

In some aspects, the environmental parameter can be a measure of an ambient chemical pollutant, such as ambient carbon dioxide, carbon monoxide, or ambient oxygen level. The environmental parameter can also be a measure of an ambient air-borne metal, ambient air quality level, ambient noise level, or a water pollutant.

The method can further comprise the step of providing the first user with at least one of an alarm and a warning, as a function of at least one of the first value and the first data reaching, exceeding or falling below a threshold value.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. In a mobile communications system that includes a cloud-based computing infrastructure and a plurality of mobile communications devices with attached sensor-based apparatus, a method for testing and monitoring chemical compositions and environmental conditions, the method comprises the steps of:
physically sensing chemical compositions or environmental conditions by one or more of the plurality of communications devices to produce information about the sensed chemical level or environmental condition;
capturing the location where the physical sensing was obtained and time the physical sensing was obtained through the use of location based services with the mobile communications devices;
augmenting the physical sensing data and information with imagery or sound recordings obtained from the mobile communications device;
augmenting the physical sensing data with human generated input, either at the time of capturing the sensing data or afterwards through use of software prompting;
validating the captured physical sensing data, location data or other environmental data through use of software prompting to the human or through use of software analytics of the physical sensing data and augmenting data;
providing, over a mobile communications system and associated networking infrastructure, the physical data and information about the sensed chemical compositions or environmental conditions to a cloud-based computing infrastructure;
analyzing the physical data and information about the sensed chemical compositions or environmental conditions to determine a recommendation for correcting detected adverse conditions by analyzing large amounts of the physical data captured over time; and
providing at least a selected portion of the analyzed data and information to at least one user.

2. The method of claim 1, further comprising the steps of:
enhancing the physical sensing capabilities of commonly available mobile communications devices with sensor-extensible attached apparatus;
capturing large amounts of physical data, including chemical compositions or environmental conditions, through the use of individual users or crowd-sourcing techniques;
augmenting the physical sensing data with human generated input, at a different time and location that the data is captured through use of software prompting;
storing, processing and analyzing of the obtained physical data and augmented information within a cloud-computing environment;
enhancing or otherwise augmenting the captured physical data and information by inserting or otherwise comparing the physical data and information with data obtained from outside sources such as government websites;
providing specific general alarms, reports or warnings to one or more users based on government standards;
providing general alarms, reports and warnings to the public affected by adverse changes in local environments, based on government standards;
providing customized general alarms, reports and warnings to interested users or groups of users affected by adverse changes in local environments, based on the user's specific areas of interests and preferences;
providing predictions of environmental conditions and physical data based on extrapolations of the large amounts of data captured by users and sensor-based communications devices over time; and
providing recommendations for techniques or processes to correct adverse conditions.

3. A method of crowd-sourcing data for an environmental parameter, using first and second hand-held mobile devices of first and second human users, respectively, comprising:
providing a first sensor to the first human user, the first sensor configured to sense a first value for the environmental parameter;
providing the first mobile device with software that receives the first value, and communicates to a data repository: (a) first data derived from the first value; (b) first location information corresponding to the first value; and (c) at least one of a first image and a first sound recording corresponding to the first value;
providing a second sensor to the second human user, the second sensor configured to sense a second value for the environmental parameter; and
providing the second mobile device with software that receives the second value, and communicates to the data repository: (a) second data derived from the second value; (b) second location information corresponding to the second value; and (c) at least one of a second image and a second sound recording corresponding to the second value.

4. The method of claim 3, wherein the step of providing the first sensor to the first human user comprises directly or indirectly selling the first sensor to the first human user.

5. The method of claim 3, wherein the location information corresponding to the first value is current within five minutes of the first mobile device using the software to receive the first value.

6. The method of claim 3, wherein at least one of the first image and the first sound recording is current within five minutes of the first mobile device receiving the first value.

7. The method of claim 3, wherein the first mobile device communicates the second data to the data repository within five minutes of the first mobile device receiving the first value.

8. The method of claim 3, wherein the data repository is distal to the first mobile device when the first mobile device communicates the first value to the data repository.

9. The method of claim 3, further comprising assisting a third-party to use information stored in the data repository to provide the first human user with a report that characterizes a health aspect of an environment of the first human user as a function of at least one of the first value and the first data.

10. The method of claim 3, further comprising directly or indirectly sending to the first human user a report that characterizes a health aspect of an environment of the first human user as a function of at least one of the first value and the first data.

11. The method of claim 10, wherein the report additionally characterizes the health aspect of the environment of the first human user as a function of (a) the first location information and (b) at least one of the first image and the first sound recording.

12. The method of claim 3, further comprising directly or indirectly sending to the first human user a personal history report that depicts time variance of a health aspect of an environment of the first human user as a function of at least one of the first value and the first data.

13. The method of claim 3, wherein the environmental parameter is a measure of an ambient chemical pollutant.

14. The method of claim 3, wherein the environmental parameter is a measure of an ambient carbon dioxide or carbon monoxide level.

15. The method of claim 3, wherein the environmental parameter is a measure of an ambient oxygen level.

16. The method of claim 3, wherein the environmental parameter is a measure of an ambient air-borne metal.

17. The method of claim 3, wherein the environmental parameter is a measure of an ambient air quality level.

18. The method of claim 3, wherein the environmental parameter is a measure of an ambient noise level.

19. The method of claim 3, wherein the environmental parameter is a measure of a water pollutant.

20. The method of claim 3, further comprising providing the first user with at least one of an alarm and a warning, as a function of at least one of the first value and the first data reaching, exceeding or falling below a threshold value.
